(19) United States
(12) Patent Application Publication
(10) Pub. No.: US 2024/0091141 A1
(43) Pub. Date: Mar. 21, 2024

(54) METHODS AND COMPOSITIONS FOR THE ABLATION OF NERVES

(71) Applicant: TULAVI THERAPEUTICS, INC., Los Gatos, CA (US)

(72) Inventors: Tim S. Jarrett, Los Gatos, CA (US); Mike Bassett, Los Gatos, CA (US); Corinne Bright, Los Gatos, CA (US); Yong Ren, Los Gatos, CA (US)

(21) Appl. No.: 18/477,383

(22) Filed: Sep. 28, 2023

Related U.S. Application Data
(63) Continuation of application No. PCT/US2022/022623, filed on Mar. 30, 2022.
(60) Provisional application No. 63/168,144, filed on Mar. 30, 2021.

(51) Int. Cl. A61K 9/00; A61K 9/06; A61K 45/06

(52) U.S. Cl. CPC A61K 9/0024; A61K 9/06; A61K 45/06

(57) ABSTRACT

Methods, devices and materials are for a pharmaceutically acceptable implant system. The implant system may comprise a neuromodulating agent, solid particulate hydrogel particle, a carrier medium in a different physical phase, and in situ formation of a singular depot for sustained release of said agent.

### INCORPORATION BY REFERENCE TO ANY PRIORITY APPLICATIONS

This application is a continuation of PCT App. No. PCT/US2022/022623, filed on Mar. 30, 2022, which claims the benefit under 35 U.S.C. § 119(e) as a nonprovisional application of U.S. Provisional App. No. 63/168,144, filed on Mar. 30, 2021, which is hereby incorporated by reference in its entirety.

### BACKGROUND

### Description

Chronic knee osteoarthritis (OA) is one of the most prevalent diseases and forms of arthritis developed in advanced age populations. Late stages of the disease are characterized by irreversible damage to articular cartilage, and hyaline articular cartilage loss representing the signature moment in the degenerative cascade to irreversible joint damage.

Rates of the disease are expected to only rise with an expanding senior population, with 10% of men and 13% of women over the age of 60 experiencing symptoms. Manifestation of clinical symptoms, such as pain, or restricted movement, can be debilitating. Patients with advanced states of the disease may seek total knee arthroplasty (TKA), but many patients are not good candidates as they experience high states of pain without loss of knee functionality. Alternatively, other patients may benefit from surgery but have comorbidities that make them poor candidates for surgery, have a fear of post-surgical pain and rehabilitation.

Current standard of care for treatment of moderate to severe osteoarthritis includes oral medication, various injectable solutions, and homeopathic solutions. Current solutions for the treatment of pain associated with OA fall short. Sustained use of oral analgesics such as NSAIDs has been associated with severe side effects such as gastrointestinal bleeding and ulcers.

Several intraarticular injection based solutions exist, often performed by an orthopedic surgeon. Intraarticular injections of hyaluronic acid (HA), also known as viscosupplementation, work based on the theory that HA is naturally found in the knee as a key component of synovial fluid, therefore addition of either large molecular weight or partially cross-linked alternatives should improve deteriorating conditions. Several products exist, including Euflexxa®, Gel-One®, Hyalgan®, Hyalgan LL®, Monovisc®, Orthovisc®, Supartz®, and Supartz FX®. However, intraarticular injection efficacy is short term if effective at all. A large meta-analysis of the impact of HA injections, covering over 12,000 patients, demonstrated the hyaluronic acid injections had either no effect or a “clinically irrelevant effect”. Evaluations of clinical and preclinical studies on the effectiveness of HA determined the beneficial effects as inconclusive. The benefits of corticosteroid injections have also been inconclusive and not shown to be statistically significant. In studies demonstrating a benefit, the use of corticosteroids had short term gains. Physicians must consider the lack of conclusive evidence when weighing cost benefit ratios to patients receiving these injections.

Alternatively, corticosteroid injections operate on the principle that reduced inflammation in the joint space will reduce the pain associated with osteoarthritis. Products include FDA-approved sterile injections of methylprednisolone acetate, triamcinolone acetate, betamethasone acetate and betamethasone sodium phosphate, triamcinolone hexacetonide, and dexamethasone. Alternative therapies such as acupuncture have also been administered for relief of pain from OA. Intraarticular injections are predominantly administered via an orthopedic surgeon.

More recently, a growing body of clinical data supports thermal nerve ablation of the sensory (genicular) nerves innervating the knee to treat chronic knee pain. The clinical data in reducing knee pain in patients both pre- and post-operatively is compelling and thus the procedure is gaining acceptance in the clinical community. The most efficacious approach is using heat, or radiofrequency ablation, to ablate the nerves; a cooling approach, cryoneurolysis is also in clinical development. Cooled-radiofrequency (RF) ablation offers the advantage of a larger zone of ablation from the transducer tip compared to conventional radiofrequency ablation, allowing for more energy and greater nerve ablation efficacy. The company Avanos offers its COOLIEF system, an example of a cooled RFA technology. Clinical data has demonstrated that greater than 70% of patients achieve 50% or more reduction in pain in comparative clinical trials.

The other thermal ablation technique that has been explored for temporary nerve ablation that has been adapted for the treatment of knee pain is cryoablation, under development by Pacira Pharmaceuticals/Myoscience called Iovera®. This operates on the principle of ablation through freezing, with a repeated local, delivery of a needle through which liquid nitrous oxide passes to cool the nerve. Each of the technologies has demonstrated clinical efficacy with a noted cost-effective benefit to the patient.

Despite the promising efficacy of these approaches, the efficacy cannot compare with the pain relief achieved with a local injection of anesthetic to the same location. The efficacy of radiofrequency approaches is highly variable and physicians cannot predict patient outcomes as a result of the therapy. As a result, physicians compensate by delivering longer or multiple thermal burns to the region to try to improve the efficacy of the procedure. Although local anesthetic may be delivered preoperatively, these procedures are painful to the patient and are not tolerated by all patients. Finally, risks associated with vascular injury after radiofrequency ablation have been reported.

Another challenge for patients with knee pain is that the pain relief for these approaches is not immediate.

However, while local anesthetic injections to the genicular nerves are highly effectively and repeatable at temporarily eliminating knee pain in these patients, radiofrequency ablation results result in highly variable reduction in pain. As a result, more repeatable and reliable results are desirable patient populations exist where outcomes of persistent chronic pain exist for patients having already undergone TKA procedures, resulting from inadequate post-surgical treatment regimens or comorbidities predisposed to unfavorable outcomes in reconstructive surgery.

Additionally, approximately 10-20% of patients continue to have chronic pain after TKA procedures, defined as pain continuing for 3 months or more post-surgery. This may be a direct result of local nerve damage that was incurred as a result of the surgery, such as damage to the common peroneal nerve, or it may be a result of inflammation and nonspecific injury to the knee region causing sustained aberrant nerve signals from the site, resulting in chronic neuropathic pain.

Both technologies rely on non-specific damage to tissue, and visualization of the genicular nerves relies on fluoroscopic and/or ultrasound landmarks, such as bone and vein, to target. As a result, natural physiological variation cannot be accounted for, leaving the potential for patient dissatisfaction or worse, unintended collateral tissue and/or nerve damage. Additionally, the procedure itself carries the burden of large intra-operative pain.

### SUMMARY

In some embodiments, an injectable drug delivery depot can be capable of delivering a neurolytic agent beyond 24 hours to provide extended pain relief, translating to improved patient outcomes, patient satisfaction, and a reduction in analgesic usage in treatment regiments for the relief of chronic pain conditions. Relief may be for 6 months. In some instances, the relief extends a year or more. Nerve ablation is complex, and unlike nerve blocks used in joint replacement surgeries that target larger, more easily visualized nerve bodies, the target is typically smaller nerve ends that cannot be visualized under ultrasound. Hydrogels can serve as depots with targeted and controlled injectate distribution, capable of high drug loading for extended release of a neuromodulating agent such as a neurolytic or anesthetic formulated to higher concentrations at which neurolytic properties are realized.

Hydrogels typically have water content >50% of their mass, for example >80%, and typically >90%. In situ forming gels have the advantage of being injectable, but present a challenge to extended delivery of soluble compounds without the use of secondary encapsulation to restrict dose dumping of their therapeutic payload and extend residence time of the therapeutic. In some embodiments, a solid dispersion of drug within a solid melt mix of one or more reactive hydrogel precursors which are then formed into particulates of the desired range for enhanced injectability in a carrier solution, without clogging of the needle or loss of injectate to the needle hub, may be utilized. As the reactive components dissolve into the liquid phase, in-situ reaction occurs; this surface boundary of reactive hydrogel continues to act as a diffusion barrier to further dissolution of the solid melt reactive mix, extending the rate of hydration and slowing down diffusion of the therapeutic contained within. In some embodiments, the solid dispersion of therapeutic is a highly water soluble drug in which this reactive phase transition extends therapeutic release from the system >24 hrs. In some embodiments, the drug having lower solubility or has secondary encapsulation where this reactive phase transition reduces the burst from the system. Other embodiments can have a bimodal release, where mixtures of solubilities or soluble and secondary encapsulation are employed to achieve a high drug release onset in the first 24-72 hrs followed by extended release for days, weeks, or months. This impediment to dissolution to extend agent release may also be increased through the incorporation of a thermally cross-linked polymer such as a pluronic incorporated into the carrier medium used for injection.

The hydrogel melt particle suspension is contrast enhanced, for single or multimodal forms of detection. Due to the solid particulate nature of the formulation, a melt suspension of particles is naturally hyperechoic compared to surrounding tissue. Hyperechoic agents may be incorporated into one or more precursors either as preformed or formation of on-demand contrast agents via device prep prior to injection to make it suitable for ultrasound-guided injections. Examples include incorporation of gases or voids into the melt particles themselves, or into the surrounding carrier medium. Contrast agents may be permanent relevant to the lifespan of the device, resorbing and clearing as hydrolysis of the device progresses. Agents may be temporary in nature, rendering the suspension detectable during the injection event, but reverting to non-interfering in diagnostic follow-ups through the course of patient recovery. The hydrogel melt suspension may also be rendered radiopaque via the use of pharmaceutically acceptable injectable contrast agents as the carrier medium surrounding the hydrogel melt particles, allowing formulations to be detectable under both ultrasound (US) and fluoroscopy.

The nerve-active agent release profile may vary. In some applications, immediate release over >1 day but less than 5 days may be desired. Additional applications may require release profiles extending several months, adopting zero or first order kinetics. Other applications may call for multimodal release profiles, with a large bolus release in less than 24 hours, followed by low therapeutic or subtherapeutic doses extending 48, 72 hours to a week or greater, even months. In one form, the suspension particle release profile may be designed to support delivery of a neurolytic for denervation surrounding the joint and extended suppression of nerve regrowth following the ablation event. In either application, the release profiles may be bimodal in design, with a large rapid release up front to maximize efficacy during a defined critical range, and lower sustained release for extended therapy beyond.

Disclosed embodiments include a pharmaceutically acceptable implant system comprising a collection of solid hydrogel particles delivered through a lumen in a carrier medium. Said hydrogel particles may consist of dehydrated covalently cross-linked particles or reactive precursors combined in a molten state as to be non-reactive, having a neuromodulating agent within the plurality of the particles in the collection. The particles themselves may serve as a hyperechoic contrast agent, capable of reacting with one another upon delivery to the target tissue. The particles are delivered in situ in a solid state, creating a sustained release depot for therapeutic agents through diffusion barriers created at the interface of the hydrating front through the cross-linked gel and dissolution of the solid particulate agent, including highly water soluble therapeutic agents. In the use of a solid melt particle of reactive precursors, this diffusion barrier is more pronounced as the particle reacts at the interface, maintaining a higher percent solids with reduced porosity relative to traditional hydrogel constructs. In some embodiments, the carrier medium may be contrast medium for the purpose of visualization under medical imaging, ultrasound or fluoroscopy, or both. The carrier medium may be covalently cross-linked to the hydrogel or physically cross-linked in the implantable materials that are injectable into the space(s) between one or more tissues in and around nerves. These injectable slurries, containing microparticles under medical device visualized injection for the localized delivery of a neuromodulating or nerve-affecting agent for the treatment of neurological disorders.

### BRIEF DESCRIPTION OF THE DRAWINGS

FIG. 1 illustrates an embodiment of a syringe comprising a pharmaceutically acceptable implant system.

### DETAILED DESCRIPTION

Materials, methods, and uses are set forth herein for a hyperechoic depot comprising a hydrogel, hyperechoic agent, and a therapeutic for use in ultrasound or fluoroscopy guided local nerve modulation. Compositions and methods are provided to create an injectable, hyperechoic and/or radiopaque depot with controlled particle distribution consisting of suspended solid reactive hydrogel precursors for placement in interstitial space in one or more tissues for the treatment of pain. Methods of using any compositions described herein in conjunction with therapeutic systems for the treatment of chronic pain are also provided.

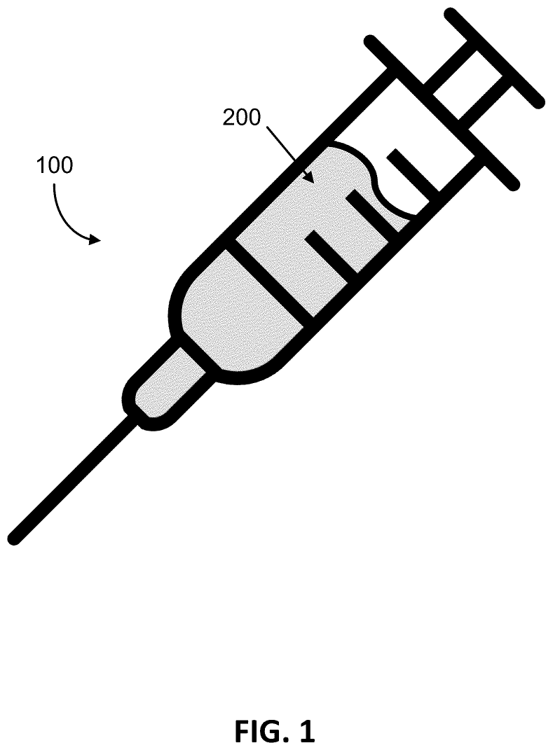

An injectable, pharmaceutically acceptable implant system may comprise a collection of pharmaceutically acceptable, reactive hydrogel precursor particles with enhanced medical imaging properties capable of being delivered in image-guided injection procedures for sustained local delivery of a therapeutic agent for the treatment of chronic pain. This would be achieved through injection between one or more tissues in proximity to a nerve with flowable, reactive hydrogel precursor solid particles to promote in-situ polymerization of the hydrogel matrix containing a therapeutic agent for relief of chronic pain conditions. The hydrogel may be echogenically enhanced by incorporation of hyperechoic agent into the reactive precursor particles, the injection carrier, or formation of on-demand hyperechoic agents via device prep prior to injection to make it suitable for ultrasound-guided injections. The hydrogel may have enhanced radiopacity by incorporation of radiopaque agent into the reactive precursor particles, or by the use of contrast agents as a carrier to make it suitable for injections under fluoroscopy. FIG. 1 illustrates an example of a syringe 100 comprising any example of a pharmaceutically acceptable implant system 200. The pharmaceutically acceptable implant system 200 may comprise any system as described herein.

One embodiment of an implant involves injecting an interspace with flowable particles consisting of a solid suspension of precursors that react to make a hydrogel implant. The precursors may contain preformed imaging agents, or the ingredients to form imaging agents on dissolution of the reactive precursors. The solid precursor particles may contain a therapeutic with neuromodulating capabilities. In some embodiments, the neuromodulating effect is a neurolytic effect capable of ablating a target nerve. A process for making the implant involves dissolution of reactive precursors at the interface of the solid particle within the surrounding aqueous carrier medium and moisture available in the injected tissue. When they react with each other, the precursors form a swellable hydrogel at the interface of solid and liquid phases that provides a diffusion barrier to further dissolution of more reactive components, with an overall effect of slowing the precursor particle dissolution and depressing release kinetics of the therapeutic from the suspension. In some embodiments, particles may be made completely of a single reactive component, then physically mixed with particles comprising a second or more reactive precursor to for a reactive precursor particle suspension capable of forming a cohesive in suit gel depot on injection.

In some embodiments, the site may be filled with small particles that are small so that they flow easily into the site. The particles are capable of injection through small cannulas, possessing controlled distribution that can be manually manipulated for some limited time after placement, and later form a cohesive singular depot or aggregation of larger depot to deter migration. Depots of reacted small particles that are pliable after reacting may be well suited as depots in periarticular regions where movement is high. Larger depots formed from reactive precursor particles may be suited to serve as an additional physical barrier to nerve regeneration that an incohesive hydrogel particle slurry would not.

In some embodiments, a therapeutic agent is incorporated to provide local delivery to a nerve in the form of a block or a neurolytic. Therapeutics may be suspended or pre-formed microparticles containing the therapeutic agents may be dispersed in the reactive solid melt reactive precursor particles. The therapeutic agent may be delivered in the form of the solid reactive precursors or incorporated into the carrier medium surrounding the solid precursors, or both.

In some embodiments, medical imaging contrast agents may be included with the implants, either previously incorporated into the precursor particles or mixed into the carrier medium surrounding the particles. In some instances, a system may consist of small particles formulated in a solution of a surfactant or emulsion agent that allows for formation of hyperechoic agents under mixing conditions prior to use. The formation of these agents may be temporary in nature, serving to visualize the injection event and later resorbing over time as to prevent interference with diagnostic follow-ups at future visits. In some forms, the particle suspension itself may serve as the hyperechoic agent. In some embodiments, the carrier medium is comprised of a pharmaceutically acceptable injectable contrast agent or is combined with a contrast agent for visualization under fluoroscopy. In some embodiments, the system is both echogenic and radiopaque for visualization under multiple medical modalities.

### Agents for Hydrogel Imaging

Other hydrogel applications would be facilitated if the particles included contrast enhancing agents for use in medical imaging procedures. The high water content of hydrogels, a key element to their biocompatibility, is also a reason they are poorly visualized under ultrasound and fluoroscopy. Particles possessing differing density relative to the surrounding tissue will increase visualization of the solid particles. Agents may also be incorporated into the carrier medium used to inject the particles. Agents, gas or solid, may be mixed with the reactive precursors during melt formation, or formulated in such a fashion that they can be generated on mixing prior to injection. In some embodiments, a biological gas, such as nitrogen, oxygen or carbon dioxide, may be entrapped within a lipid bilayer to form a stable microbubble suspension to be suspended within the melt used to form the solid particles. Other embodiments may use artificial gases, such as perfluorocarbons, with reduced solubility in aqueous environments. Certain gas filled liposomes are known in the medical arts, e.g., as used in products such as Optison™, Levovist™, or Sonazoid.

Some embodiments may incorporate of aqueous surfactants, such that microbubbles, may be formed “on demand” with the methods of the solid reactive hydrogel precursor particles with a fluid carrier medium for injection. Aqueous miscible agents such as non-ionic surfactants may be used alone or in combination to produce microbubbles after manually mixing one or more formulation precursors between two syringes prior to injection. In some embodiment, these agents are used to produce microbubbles in precursors via mechanical constructs in the application device, such as a modified syringe and/or static mixer. In further embodiments, an external gas source is supplied as part of the application device, the gas source pressurized as such to provide the kinetics for microbubble formation in the carrier medium. Additionally, predissolved reactive components may be separated within one or more formulation precursors such that the combination of the precursors causes an effervescent reaction, producing microbubbles. One embodiment involves formulating one or more precursors with an existing commercial microbubble formulation such as Optison™, Levovist™, or Sonazoid™. Some embodiment can include use of hydrogel particles mixed with a pharmaceutically-approved commercial contrast agent.

One process for forming contrast enhanced hydrogel particles involves incorporating density altering agents into the particle matrix. In one embodiment, this involves incorporation of gaseous agents and trapping them in the solid reactive precursor particles prior to reconstitution. In some embodiments, the density agent is a solid in suspension, where the solid is an agent capable of resorption. Bioerodable or biodegradable polymers such as polymers and copolymers of: poly(anhydride), poly(hydroxy acid)s, poly(lactone)s, poly(trimethylene carbonate), poly(glycolic acid), poly(lactic acid), poly(glycolic acid)-co-poly(glycolic acid), poly(orthocarbonate), poly(caprolactone), cross-linked biodegradable hydrogel networks like fibrin glue or fibrin sealant, caging and entrapping molecules, like cyclodextrin, molecular sieves and the like. Microspheres made from polymers and copolymers of poly (lactone)s and poly (hydroxy acid) may be used as biodegradable imaging vehicles.

Examples include blank PLLA or PLGA microparticles suspended into the carrier medium when the injectable system is formed. Alternatively, low solubility or slow dissolution polymers, such as high Mw hydrophilic polymers such as linear PEG, may be suspended in the hydrogel particles or in one or more flowable precursors to provide hyperechoic properties on injection. Reabsorbable suspension agents, either through bond hydrolysis or dissolution, have the potential advantage of providing transient contrast properties. An injectable particle depot with decreasing hyperechoic properties over time allow for visualization during placement events and reduction of interference with future diagnostic follow ups through the device lifespan. An example would be the injection of a hyperechoic hydrogel particle slurry containing solid particles of 100 kDa linear PEG. The linear PEG would dissolve slowly enough to allow for the implant procedure time (i.e. injection), then dissolve leaving the hydrogel particle depot hypogenic and therefore non-interfering to follow up ultrasounds used to evaluate the joint. In some embodiments, the suspension comprises the therapeutic or combination of therapeutics intended for extended delivery to the surrounding tissue. The echogenicity of the slurry depot decreases with time as the therapeutic is released, providing an advantageous dual functionality of visualization on implantation and a visual correlation with time to remaining therapeutic reservoir. An example would be a high loading suspension of amitriptyline particles dispersed in a solid reactive precursor particle. Degrees of echogenicity would directly correlate to the amount of suspended amitriptyline and density of the particle remaining.

Radiopacity may be achieved in some embodiments through incorporation of commercially available contrast agents. These include all aqueous based non-ionic iodinated contrast medias with an iodine content >300 mgl/mL (i.e. Omnipaque 300/350, Isovue 320, etc.).

Additional machine-aided imaging agents may be used in addition to, or as alternatives to, radiopaque and/or echogenic compounds. Such agents are, for example fluorescent compounds or MRI contrast agents (e.g., Gadolinium containing compounds).

### Drugs or Other Therapeutic Agents for Delivery

Alcohol and phenol (carbolic acid, monohydroxybenzene) are both commonly used neurolytic agents. Alcohol causes an immediate progressive burning paresthesia that lasts several hours but a wide range of ethanol concentrations are effective at destroying nerves through extraction of cholesterol and phospholipids and subsequent sclerosis. Concentrations above 50% are well established to result in neurolysis, such as about 75%, 80%, 99% or 100%. One-hundred percent ethanol has been demonstrated to completely destroy the cell bodies and axons of sympathetic, sensory and motor neurons but come with a higher risk of adjacent neuritis. Phenol has mild anesthetic properties and causes a focal hemorrhagic necrosis and dissolves axons and Schwann cells inside the basal lamina, resulting in damage to the entire endoneurium. Regeneration in the periphery may begin in 2 weeks in preclinical studies. The drug can be injected at, for example, between 3 and 10%, more typically between 6.7% to 7% in oil or glycerol, such as Phenol-Aqua (7%) or phenol-glycerol (5%). Higher concentrations have been applied, such as about or at least about 10%, 25%, 50%, and 75%, such as between about 10-50% phenol in ethanol is desirable in some cases. Both produce severe burning pain immediately upon injection which may last about a minute. Glycerol is an anhydrous less toxic alcohol with weaker penetration, less extensive neuronal damage and faster regeneration than alcohol and phenol. Iohexol (30%) may also be employed. Alternatively, sodium tetradecyl sulfate (STS), an anionic surfactant and sclerosant drug with detergent properties may be selected.

Norepinephrine reuptake inhibitors (NRIs) and less specific norepinephrine serotonin reuptake inhibitors (SNRIs) (and selective serotonin/5-hydroxytryptamine reuptake inhibitors (SSRIs) and dopamine reuptake inhibitors) block the reuptake of norepinephrine at the synaptic cleft thereby increasing and sustaining the action of norepinephrine at the nerve terminal in the heart and other tissues. Norepinephrine uptake transporters (NET) includes Uptake 1, present in the neurons and lung pulmonary endothelial cells and uptake 2 transporter, present in the myocardium. Reuptake inhibitors include guanethidine, 1-methyl-4-phenyl-pyridinium ion (MPP+) and Oxidopamine or 6-hydroxydopamine (6-OHDA), alpha-methyldopa, bretylium tosylate, guanacline, bethanidine and debrisoquine, desipramine, nisoxetine, ritanserin, setoperone, volinanserin, duloxetine, citalopram, fluvoxamine, zimeldine, sibutramine, Levomilnacipran, debrisoquine, lobeline and amezinium. Dopamine reuptake inhibitors include GBR-12909 and amfonelic acid. Many of these agents also function as MAO inhibitors to prevent norepinephrine deamination and some as a VMAT agonist. Although not a reuptake inhibitor, alkaloid cocaine interferes with Uptake-1. Guanethidine (1-2 mg/ml) is particularly interesting in some embodiments because it can both increase the norepinephrine in the synaptic cleft (transient sympathomimetic) initially through NET1 activity but also acting as a monoamine depleting agent, and blocks adrenergic transmission. High or sustained doses lead to neuronal cell death in both efferent and afferent nerves, such as capsaicin-sensitive primary sensory nerves. Preferably, these agents are delivered to nerve terminal or peripheral synapse of the post-ganglionic sympathetic nerve in the heart, lung, or tissue innervated by post-ganglionic sympathetic efferent nerves. At high concentrations, these agents result in immunotoxic NK- and mononuclear-cell mediated death as can be seen by degeneration of sympathetic ganglia in the sympathetic chain.

### Anti-Depressants.

The neuromodulatory agent may be an anti-depressant such as bupropion, doxepin, desipramine, clomipramine, imipramine, nortriptyline, amitriptyline, protriptyline, trimipramine, tianeptine, fluoxetine, fluvoxamine, paroxetine, sertraline, phenelzine, tranylcypromine, amoxapine, maprotiline, trazodone, venlafaxine, mirtazapine, their pharmaceutically active salts and/or their optical isomers. In a very preferred embodiment, the anti-depressant is either bupropion or a pharmaceutically acceptable salt thereof, or nortriptyline or a pharmaceutically acceptable salt thereof. Bupropion, desipramine and imipramine are also ganglionic blocking agents (nicotinic) and at higher doses is toxic to afferent and efferent nerves.

Microtubule disrupting agents or cytoskeletal drugs that interact with actin or tubulin may also be used to denervate neurons such as phalloidin, cytochalaisin D, Latrunculin, colchicine (1 and 10 microM), demecolcine, jasplakinolide, nocodazole, paclitaxel (taxol), and vinblastine. Other potential approaches include inhibition of phophoinositide 3-kinase (PI3K), serine-threonine protein kinase B (Akt), extracellular signal-regulated kinase (ERK) pathway, the P38 mitogen activated protein kinase pathway (MAPK).

Cholesterol oxides (PMID 9566506) cause rapid cell sympathetic ganglia cell death in vitro at concentration of 4 ug/ml (10 uM) within 36 hours. The most potent of these 25-OH-cholesterol has demonstrated neurotoxicity across a range of cell types.

MAO-A and COMPT inhibitors, including tyramine, clorgyline, paragyline and 3,5-dinitrocatechol, Ro 41-1049, selegiline, tranylcypromine may result in excitatory chemical sympathectomy if delivered in high enough levels.

Immunosympathectomy can be achieved with Anti-Nerve growth Factor (anti-NGF, Tanezumab, Fulranumab), auto-immune sympathectomy with Anti-Dopamine Beta Hydroxylase (DHIT), DBH or Anti-acetylcholinesterase (Anti-AChE, immunotoxin sympathectomy with OX7-SAP, 192-SAP IgG, DBH-SAP or DHIT. Toxins such as botulinum toxin (BOTOX, DYSPORT type A through G, such as described, for example, in U.S. Pat. No. 6,743,424 to Donovan, which is hereby incorporated by reference in its entirety), tetrodotoxin, neosaxitoxin, may also be effective.

### Amino Amides

The neuromodulating agent may be an anesthetic. Anesthetics operate to block voltage-gated sodium channels, which prevents sodium influx into the cell and blocks impulse transmission. Local anesthetics are also class I antiarrhythmic drugs due to the blockade of cardiac sodium channels, with lidocaine being the class IB prototype. They selectively block channels that are frequently depolarizing (tachyarrhythmias) and slow transmission. Anesthetics, at high local concentrations, have been shown to possess neurolytic properties.

Two subclasses of local anesthetics categorize according to the location where metabolism occurs, the amino amides and amino esters. The amino-amides such as bupivacaine, ropivacaine, and lidocaine, are hydrolyzed in the liver. Amino-esters are prone to allergic responses and lack solution stability. Amino-amides

### Therapeutic Delivery

In some embodiments described above and herein, the nerve-active agent or agents are formulated into a mixture of molten reactive precursors, which when solidified, contains the agent as a dispersed suspension through the reactive precursor solid matrix. The agent may be a solid core with a molten reactive precursor shell.

In certain embodiments the agent is suspended as native drug. The agent may be in a plurality of hydrophobic or hydrophilic domains, in a separate phase, either as complex or as a mixture comprising agent and domain. An example is a solid particle of bupivacaine entrapped within a liposome, isolated and then introduced into a molten mixture of reactive precursors. Another example is amiodarone, complexed in a solution with beta-cyclodextrin, dried, isolated and then incorporated into a melt mixture of reactive hydrogel precursors. Mixtures would be incorporated in methods described above, either in one or more precursors, or incorporated into a hydrogel matrix that is further reduced to a flowable slurry.

In some embodiments described above and herein, the nerve-active agent is formulated for immediate release in >24 hrs but less than 72 hours. Additional embodiments may be formulated for release <1 week. Additional embodiments may include formulating for 1 week, 1 month, 3 months, 6 months, 9 months, 1 year. In extended release embodiments, release rate profiles may take on zero, first or second order kinetic curves. Other embodiments of release profile have characteristics of second order release for one part of their profile, zero order for a second part, and first order kinetic characteristics for the third. Examples of these include therapeutic agents encapsulated in polyester systems such as PLA, where the mechanisms of swelling, bulk erosion and diffusion of the drug compete in different various degrees as the particle hydrolyzes. Reactive precursor particles containing agents with secondary encapsulation such as PLA microparticles may be formed to achieve such release rates. Additionally, blends of multiple release profile through a plurality of particles may be performed to achieve a different release rate kinetics. Other embodiments may call for multimodal release profiles, with a large bolus release in the first 48-72 hours followed by low therapeutic or subtherapeutic doses extending through the desired window of efficacy. In one embodiment, multimodal release rates are created from any number of combinations of the rates described above.

In one example, a reactive particle suspension containing 15% (wt) amitriptyline was formulated to have <40% burst in 1 hr but NMT 80% release in 24 hrs. Herein, desired release profile therapy times are defined as NLT 80% w/w of the nerve active agent released in said time.

### Particles

Embodiments may include the addition of an osmotic agent to a plurality of particles. Examples of such agents include salts and polymers. Embodiments include polymers, linear polymers, and hydrophilic polymers, or combinations of the same. Embodiments include polymers of between about 500 and about 100,000 molecular weight; artisans will immediately appreciate that all the ranges and values within the explicitly stated ranges are contemplated, e.g., about 5000 to about 50,000 molecular weight. Embodiments include, for example, a concentration of about 1% to about 50% w/w osmotic agent; artisans will immediately appreciate that all the ranges and values within the explicitly stated ranges are contemplated, e.g., 10% to 30%. The agent and hydrogel may be introduced into a patient and may be part of a kit for the same.

Some embodiments of the system include linear hydrophilic polymers for the purpose of repairing nerves, fusing agents hereby referred to as fusogens. All eukaryotic cells (including neurons) seal plasmalemmal damage by Ca2+-dependent production of vesicles that form a plug, often at a partially-constricted cut end of a severed nerve. The Ca2+ influx activates proteins, vesicle accumulation and fusion, and biochemical pathways that enable neurons and other cells to seal membrane damage, stop Ca2+ influx, and thereby survive. In contrast, PEG does not use any of the reported pathways to rapidly and artificially fuse plasmalemmal damage. It is hypothesized that PEG directly induces membrane fusion by dessicating closely apposed membranes, thereby allowing membrane lipids to collapse and fuse cut ends. In one embodiment, the hydrogel system containing a linear osmotic agent may be used and applied to severed nerve ends to act as a fusogen for nerve repair. In some embodiments, the presence of a linear osmotic agent such as PEG in situ hydrogel depot may be used to seal a transected nerve and promote fusion of proximal and distal aspects of said nerve.

In some embodiments described above and herein, the hydrogel particles contain more than one phase, in particular a gas, biological or synthetic. The resulting particle slurry has particular properties such as reduced density or compressibility that are advantageous for particular applications. Applications may include filling of conformal spaces around articulating anatomy that solid gel implants would present challenges with foreign body, agitation, irritation or inflammation.

### Total Knee Arthroplasty (TKA)

TKA is presently one of the most common orthopedic surgeries performed in the United States, with up to 700,000 surgeries annually. With an aging population, the projections are expected to exceed 3M by 2030. A prospective cohort study of 4709 patients following total joint replacement determined that the two most important factors affecting patient satisfaction were meeting pre-operative expectations and pain relief.

### Extended Relief for Osteoarthritis

Arthroplasty is an effective and established terminal therapeutic option for late-stage osteoarthritis-related pain and dysfunction; however, the procedure may not be appropriate in all patients due to co-morbidities, lack of social support, or other factors. In light of the recent opioid crisis, alternatives have been sought for patients who do not qualify for TKA, or the estimated 20% of TKA patients with a continuum of post-operative pain.

Patients with chronic knee pain that respond positively to local genicular nerve blocks with a local anesthetic may be viable for ablation. The genicular nerves targeted for ablation include the superior laterial, the superior medial and the inferior medial nerves. These 3 sensory nerves are thought to be primarily responsible for transmitting nociceptive pain signals from the knee to the brain. Ablation that is performed correctly should cause iatrogenic neural degeneration of these nerves without motor deficits.

Chemical ablation can be performed by injecting agents such as ethanol near and around the genicular nerves to cause ablation. The issue with many chemical ablation compounds is the dependency on cellular toxicity and non-specificity in their mode of action. Compounds such as ethanol and phenol successfully ablate nerves, but side effects include pain, transient fever and potential intoxication. It is ever more important with nonspecific chemical ablation agents that the target nerve be visualized for direct injection to prevent unwanted toxicity to surrounding tissue, further confounding the use of chemical agents in genicular nerve ablation.

Radio frequency ablation (RFA) involves the production of a heat lesion via electricity conducted through an electrode catheter tip. To improve RFA outcomes, newer forms apply cooling to the tip of the cauterizing catheter to allow for larger area effects. The targets of genicular nerve ablation are not actually visualized intra-operatively; instead targets are approximated based on nearby anatomical landmarks (i.e. bone). Cooled RFA is believed to have improved efficacy as a result of overcoming anatomical differences between patients.

Though RFA offers some improvement relative to chronic intracapsular corticosteroid injections or oral opioid use, it is not without its issues. Intra-operative pain due to insertion of ablation probes and ablation itself is reported as high. Efficacy of pain relief drops with repeat application. The production of a larger lesion using cooling technology worries some that additional collateral tissue damage will occur.

Embodiments may include the infiltration of reactive hydrogel particle suspension loaded with an extended release neurolytic in and around genicular nerve targets to ablate and provide extended relief for chronic pain to the joint. An example of this would be the injection of a radiopaque hydrogel particle slurry containing high loading of bupivicaine in superior laterial, the superior medial and the inferior medial nerves under fluorscopy. In one embodiment, the gel particle slurry acts to contain the injectate and limit dispersion of the neurolytic to prevent unwanted collateral damage to surrounding tissue and/or non-target nerves.

In some embodiments, the hydrogel particle slurry is loaded with a nerve-acting agent that released in a bimodal fashion. The first release profile is rapid, 80% released within 48 hours. In some instances, 80% is released in 72 hours. At this therapeutic concentration, the activity of the agent is neurolytic in nature, and ablates the genicular nerve. The second profile is more sustained and has a lower pharmacokinetic Cmax but a greater AUC, providing sustained neurosuppression at this therapeutic level and pain relief after the ablation event. Agents such as lidocaine and bupivacaine may exhibit these dual effects at differing concentration profiles. An example of this would be a solid reactive hydrogel precursor particle suspension containing a plurality of particles of one or more populations coated in Tween80/Span 60, comprising air cores, bupivacaine HCl, and bupivacaine freebase. The bupivacaine HCl will provide the instant ablative concentrations required within the first 48 hours, while the free base will provide sustained lower analgesic therapy for months depending on formulation technique.

Post Joint Arthroplasty Applications

Other clinical procedures include Hip replacements, but replacement surgery can be performed on other joints, as well, including the ankle, wrist, shoulder, and elbow. In one embodiment, the hydrogel particle slurry containing imaging and nerve-acting therapeutic may be used to perform blocks on groups innervating the specific target joint. In some instances, the slurry with therapeutic may be used to ablate the sensory nerves innervating the specific target joint. In some embodiments, the suspension is used to ablate nerves following total joint replacement that continues to suffer from chronic pain.

### Other Clinical Needs

### Hydrogels

Accordingly, embodiments are provided herein for making implant materials. Such materials include matrices with a porosity of more than about 20% v/v; artisans will immediately appreciate that all the ranges and values within the explicitly stated range is contemplated. Hydrogels are an embodiment of such an implant. Hydrogels are materials that do not dissolve in water and retain a significant fraction (more than 20%) of water within their structure. In fact, water contents may be in excess of 90%. Hydrogels are often formed by crosslinking water soluble molecules to form networks of essentially infinite molecular weight. Hydrogels with high water contents are typically soft, pliable materials. A hydrogel that has been dried is referred to herein as a dehydrated hydrogel if it will return to a hydrogel state upon exposure to water; this hydrogel would expand in volume if it were exposed to an excess of water and not constrained. The term desiccated refers to a hydrogel essentially having no fluids, bearing in mind that some trace amounts of water may nonetheless be present.

Hydrogels may be formed from natural, synthetic, or biosynthetic polymers. Natural polymers may include glycosminoglycans, polysaccharides, and proteins. Some examples of glycosaminoglycans include dermatan sulfate, hyaluronic acid, the chondroitin sulfates, chitin, heparin, keratan sulfate, keratosulfate, and derivatives thereof. In general, the glycosaminoglycans are extracted from a natural source and purified and derivatized. However, they also may be synthetically produced or synthesized by modified microorganisms such as bacteria. These materials may be modified synthetically from a naturally soluble state to a partially soluble or water swellable or hydrogel state. This modification may be accomplished by conjugation or replacement of ionizable or hydrogen bondable functional groups such as carboxyl and/or hydroxyl or amine groups with other more hydrophobic groups.

For example, carboxyl groups on hyaluronic acid may be esterified by alcohols to decrease the solubility of the hyaluronic acid. Such processes are used by various manufacturers of hyaluronic acid products (such as Genzyme Corp., Cambridge, Mass.) to create hyaluronic acid based sheets, fibers, and fabrics that form hydrogels. Other natural polysaccharides, such as carboxymethyl cellulose or oxidized regenerated cellulose, natural gum, agar, agrose, sodium alginate, carrageenan, fucoidan, furcellaran, laminaran, hypnea, eucheuma, gum arabic, gum ghatti, gum karaya, gum tragacanth, locust beam gum, arbinoglactan, pectin, amylopectin, gelatin, hydrophilic colloids such as carboxymethyl cellulose gum or alginate gum cross-linked with a polyol such as propylene glycol, and the like, also form hydrogels upon contact with aqueous surroundings.

Synthetic hydrogels may be biostable or biodegradable or biodegradable. Examples of bio stable hydrophilic polymeric materials are poly(hydroxyalkyl methacrylate), poly(electrolyte complexes), poly(vinylacetate) cross-linked with hydrolysable or otherwise degradable bonds, and water-swellable N-vinyl lactams. Other hydrogels include hydrophilic hydrogels known as CARBOPOL®, an acidic carboxy polymer (Carbomer resins are high molecular weight, allylpentaerythritol-crosslinked, acrylic acid-based polymers, modified with C10-C30 alkyl acrylates), polyacrylamides, polyacrylic acid, starch graft copolymers, acrylate polymer, ester cross-linked polyglucan. Such hydrogels are described, for example, in U.S. Pat. No. 3,640,741 to Etes, U.S. Pat. No. 3,865,108 to Hartop, U.S. Pat. No. 3,992,562 to Denzinger et al., U.S. Pat. No. 4,002,173 to Manning et al., U.S. Pat. No. 4,014,335 to Arnold and U.S. Pat. No. 4,207,893 to Michaels, all of which are incorporated herein by reference, with the present specification controlling in case of conflict.

Hydrogels may be made from precursors. The precursors are not hydrogels but are covalently cross-linked with each other to form a hydrogel and are thereby part of the hydrogel. Crosslinks can be formed by covalent or ionic bonds, by hydrophobic association of precursor molecule segments, or by crystallization of precursor molecule segments. The precursors can be triggered to react to form a cross-linked hydrogel. The precursors can be polymerizable and include crosslinkers that are often, but not always, polymerizable precursors. Polymerizable precursors are thus precursors that have functional groups that react with each other to form polymers made of repeating units. Precursors may be polymers.

Some precursors thus react by chain-growth polymerization, also referred to as addition polymerization, and involve the linking together of monomers incorporating double or triple chemical bonds. These unsaturated monomers have extra internal bonds which are able to break and link up with other monomers to form the repeating chain. Monomers are polymerizable molecules with at least one group that reacts with other groups to form a polymer. A macromonomer (or macromer) is a polymer or oligomer that has at least one reactive group, often at the end, which enables it to act as a monomer; each macromonomer molecule is attached to the polymer by reaction the reactive group. Thus macromonomers with two or more monomers or other functional groups tend to form covalent crosslinks. Addition polymerization is involved in the manufacture of, e.g., polypropylene or polyvinyl chloride. One type of addition polymerization is living polymerization.

Some precursors thus react by condensation polymerization that occurs when monomers bond together through condensation reactions. Typically these reactions can be achieved through reacting molecules incorporating alcohol, amine or carboxylic acid (or other carboxyl derivative) functional groups. When an amine reacts with a carboxylic acid an amide or peptide bond is formed, with the release of water. Some condensation reactions follow a nucleophilic acyl substitution, e.g., as in U.S. Pat. No. 6,958,212, which is hereby incorporated by reference herein in its entirety to the extent it does not contradict what is explicitly disclosed herein.

Some precursors react by a chain growth mechanism. Chain growth polymers are defined as polymers formed by the reaction of monomers or macromonomers with a reactive center. A reactive center is a particular location within a chemical compound that is the initiator of a reaction in which the chemical is involved. In chain-growth polymer chemistry, this is also the point of propagation for a growing chain. The reactive center is commonly radical, anionic, or cationic in nature, but can also take other forms. Chain growth systems include free radical polymerization, which involves a process of initiation, propagation and termination. Initiation is the creation of free radicals necessary for propagation, as created from radical initiators, e.g., organic peroxide molecules. Termination occurs when a radical reacts in a way that prevents further propagation. The most common method of termination is by coupling where two radical species react with each other forming a single molecule.

Some precursors react by a step growth mechanism, and are polymers formed by the stepwise reaction between functional groups of monomers. Most step growth polymers are also classified as condensation polymers, but not all step growth polymers release condensates.

Monomers may be polymers or small molecules. A polymer is a high molecular weight molecule formed by combining many smaller molecules (monomers) in a regular pattern. Oligomers are polymers having less than about 20 monomeric repeat units. A small molecule generally refers to a molecule that is less than about 2000 Daltons.

The precursors may thus be small molecules, such as acrylic acid or vinyl caprolactam, larger molecules containing polymerizable groups, such as acrylate-capped polyethylene glycol (PEG-diacrylate), or other polymers containing ethylenically-unsaturated groups, such as those of U.S. Pat. No. 4,938,763 to Dunn et al, U.S. Pat. Nos. 5,100,992 and 4,826,945 to Cohn et al, or U.S. Pat. Nos. 4,741,872 and 5,160,745 to DeLuca et al., each of which is hereby incorporated by reference herein in its entirety.

To form covalently cross-linked hydrogels, the precursors must be cross-linked together. In general, polymeric precursors will form polymers that will be joined to other polymeric precursors at two or more points, with each point being a linkage to the same or different polymers. Precursors with at least two reactive groups can serve as crosslinkers since each reactive group can participate in the formation of a different growing polymer chain. In the case of functional groups without a reactive center, among others, crosslinking requires three or more such functional groups on at least one of the precursor types. For instance, many electrophilic-nucleophilic reactions consume the electrophilic and nucleophilic functional groups so that a third functional group is needed for the precursor to form a crosslink. Such precursors thus may have three or more functional groups and may be cross-linked by precursors with two or more functional groups. A cross-linked molecule may be cross-linked via an ionic or covalent bond, a physical force, or other attraction. A covalent crosslink, however, will typically offer stability and predictability in reactant product architecture.

In some embodiments, each precursor is multifunctional, meaning that it comprises two or more electrophilic or nucleophilic functional groups, such that a nucleophilic functional group on one precursor may react with an electrophilic functional group on another precursor to form a covalent bond. At least one of the precursors comprises more than two functional groups, so that, as a result of electrophilic-nucleophilic reactions, the precursors combine to form cross-linked polymeric products.

The precursors may have biologically inert and hydrophilic portions, e.g., a core. In the case of a branched polymer, a core refers to a contiguous portion of a molecule joined to arms that extend from the core, with the arms having a functional group, which is often at the terminus of the branch. The hydrophilic precursor or precursor portion preferably has a solubility of at least 1 g/100 mL in an aqueous solution. A hydrophilic portion may be, for instance, a polyether, for example, polyalkylene oxides such as polyethylene glycol (PEG), polyethylene oxide (PEO), polyethylene oxide-co-polypropylene oxide (PPO), co-polyethylene oxide block or random copolymers, and polyvinyl alcohol (PVA), poly (vinyl pyrrolidinone) (PVP), poly (amino acids, dextran, or a protein. The precursors may have a polyalkylene glycol portion and may be polyethylene glycol based, with at least about 80% or 90% by weight of the polymer comprising polyethylene oxide repeats. The polyethers and more particularly poly (oxyalkylenes) or poly (ethylene glycol) or polyethylene glycol are generally hydrophilic.

A precursor may also be a macromolecule (or macromer), which is a molecule having a molecular weight in the range of a thousand to many millions. In some embodiments, however, at least one of the precursors is a small molecule of about 1000 Da or less. The macromolecule, when reacted in combination with a small molecule of about 1000 Da or less, is preferably at least five to fifty times greater in molecular weight than the small molecule and is preferably less than about 60,000 Da; artisans will immediately appreciate that all the ranges and values within the explicitly stated ranges are contemplated. A more preferred range is a macromolecule that is about seven to about thirty times greater in molecular weight than the crosslinker and a most preferred range is about ten to twenty times difference in weight. Further, a macromolecular molecular weight of 5,000 to 50,000 is useful, as is a molecular weight of 7,000 to 40,000 or a molecular weight of 10,000 to 20,000.

Certain macromeric precursors are the cross-linkable, biodegradable, water-soluble macromers described in U.S. Pat. No. 5,410,016 to Hubbell et al, which is hereby incorporated herein by reference in its entirety to the extent it does not contradict what is explicitly disclosed. These macromers are characterized by having at least two polymerizable groups, separated by at least one degradable region.

Synthetic precursors may be used. Synthetic refers to a molecule not found in nature or not normally found in a human. Some synthetic precursors are free of amino acids or free of amino acid sequences that occur in nature. Some synthetic precursors are polypeptides that are not found in nature or are not normally found in a human body, e.g., di-, tri-, or tetra-lysine. Some synthetic molecules have amino acid residues but only have one, two, or three that are contiguous, with the amino acids or clusters thereof being separated by non-natural polymers or groups. Polysaccharides or their derivatives are thus not synthetic.

Alternatively, natural proteins or polysaccharides may be adapted for use with these methods, e.g., collagens, fibrin(ogen)s, albumins, alginates, hyaluronic acid, and heparins. These natural molecules may further include chemical derivitization, e.g., synthetic polymer decorations. The natural molecule may be cross-linked via its native nucleophiles or after it is derivatized with functional groups, e.g., as in U.S. Pat. Nos. 5,304,595, 5,324,775, 6,371,975, and 7,129,210, each of which is hereby incorporated by reference to the extent it does not contradict what is explicitly disclosed herein. Natural refers to a molecule found in nature. Natural polymers, for example proteins or glycosaminoglycans, e.g., collagen, fibrinogen, albumin, and fibrin, may be cross-linked using reactive precursor species with electrophilic functional groups. Natural polymers normally found in the body are proteolytically degraded by proteases present in the body. Such polymers may be reacted via functional groups such as amines, thiols, or carboxyls on their amino acids or derivatized to have activatable functional groups. While natural polymers may be used in hydrogels, their time to gelation and ultimate mechanical properties must be controlled by appropriate introduction of additional functional groups and selection of suitable reaction conditions, e.g., pH.

Precursors may be made with a hydrophobic portion provided that the resultant hydrogel retains the requisite amount of water, e.g., at least about 20%. In some cases, the precursor is nonetheless soluble in water because it also has a hydrophilic portion. In some instances, the precursor makes dispersion in the water (a suspension) but is nonetheless reactable to from a cross-linked material. Some hydrophobic portions may include a plurality of alkyls, polypropylenes, alkyl chains, or other groups. Some precursors with hydrophobic portions are sold under the trade names PLURONIC F68, JEFFAMINE, or TECTRONIC. A hydrophobic portion is one that is sufficiently hydrophobic to cause the macromer or copolymer to aggregate to form micelles in an aqueous continuous phase or one that, when tested by itself, is sufficiently hydrophobic to precipitate from, or otherwise change phase while within, an aqueous solution of water at pH from about 7 to about 7.5 at temperatures from about 30 to about 50 degrees Centigrade.

Precursors may have, e.g., 2-100 arms, with each arm having a terminus, bearing in mind that some precursors may be dendrimers or other highly branched materials. An arm on a hydrogel precursor refers to a linear chain of chemical groups that connect a crosslinkable functional group to a polymer core. Some embodiments are precursors with between 3 and 300 arms; artisans will immediately appreciate that all the ranges and values within the explicitly stated ranges are contemplated, e.g., 4 to 16, 8 to 100, or at least 6 arms.

Thus hydrogels can be made, e.g., from a multi-armed precursor with a first set of functional groups and a low molecular-weight precursor having a second set of functional groups. For example, a six-armed or eight-armed precursor may have hydrophilic arms, e.g., polyethylene glycol, terminated with primary amines, with the molecular weight of the arms being about 1,000 to about 40,000; artisans will immediately appreciate that all ranges and values within the explicitly stated bounds are contemplated. Such precursors may be mixed with relatively smaller precursors, for example, molecules with a molecular weight of between about 100 and about 5000, or no more than about 800, 1000, 2000, or 5000 having at least about three functional groups, or between about 3 to about 16 functional groups; ordinary artisans will appreciate that all ranges and values between these explicitly articulated values are contemplated. Such small molecules may be polymers or non-polymers and natural or synthetic.

Precursors that are not dendrimers may be used. Dendritic molecules are highly branched radially symmetrical polymers in which the atoms are arranged in many arms and subarms radiating out from a central core. Dendrimers are characterized by their degree of structural perfection as based on the evaluation of both symmetry and polydispersity and require particular chemical processes to synthesize. Accordingly, an artisan can readily distinguish dendrimer precursors from non-dendrimer precursors. Dendrimers have a shape that is typically dependent on the solubility of its component polymers in a given environment, and can change substantially according to the solvent or solutes around it, e.g., changes in temperature, pH, or ion content.

Precursors may be dendrimers, e.g., as in Patent Application Pub. Nos. US 20040086479, US 20040131582, WO 07005249, WO 07001926, WO 06031358, or the U.S. counterparts thereof; dendrimers may also be useful as multifunctional precursors, e.g., as in U.S. Pat. Pub. No's. US 20040131582, US 20040086479 and PCT Applications No. WO 06031388 and WO 06031388; each of which US and PCT applications are hereby incorporated by reference herein in its entirety. Dendrimers are highly ordered possess high surface area to volume ratios, and exhibit numerous end groups for potential functionalization. Embodiments include multifunctional precursors that are not dendrimers.

Some embodiments include a precursor that consists essentially of an oligopeptide sequence of no more than five residues, e.g., amino acids comprising at least one amine, thiol, carboxyl, or hydroxyl side chain. A residue is an amino acid, either as occurring in nature or derivatized thereof. The backbone of such an oligopeptide may be natural or synthetic. In some embodiments, peptides of two or more amino acids are combined with a synthetic backbone to make a precursor; certain embodiments of such precursors have a molecular weight in the range of about 100 to about 10,000 or about 300 to about 500 Artisans will immediately appreciate that all ranges and values between these explicitly articulated bounds are contemplated.

Precursors may be prepared to be free of amino acid sequences cleavable by enzymes present at the site of introduction, including free of sequences susceptible to attach by metalloproteinases and/or collagenases. Further, precursors may be made to be free of all amino acids, or free of amino acid sequences of more than about 50, 30, 20, 10, 9, 8, 7, 6, 5, 4, 3, 2, or 1 amino acids. Precursors may be non-proteins, meaning that they are not a naturally occurring protein and cannot be made by cleaving a naturally occurring protein and cannot be made by adding synthetic materials to a protein. Precursors may be non-collagen, non-fibrin, non-fibrinogen), and non-albumin, meaning that they are not one of these proteins and are not chemical derivatives of one of these proteins. The use of non-protein precursors and limited use of amino acid sequences can be helpful for avoiding immune reactions, avoiding unwanted cell recognition, and avoiding the hazards associated with using proteins derived from natural sources. Precursors can also be non-saccharides (free of saccharides) or essentially non-saccharides (free of more than about 5% saccharides by w/w of the precursor molecular weight. Thus a precursor may, for example, exclude hyaluronic acid, heparin, or gellan. Precursors can also be both non-proteins and non-saccharides.

Peptides may be used as precursors. In general, peptides with less than about 10 residues are preferred, although larger sequences (e.g., proteins) may be used. Artisans will immediately appreciate that every range and value within these explicit bounds is included, e.g., 1-10, 2-9, 3-10, 1, 2, 3, 4, 5, 6, or 7. Some amino acids have nucleophilic groups (e.g., primary amines or thiols) or groups that can be derivatized as needed to incorporate nucleophilic groups or electrophilic groups (e.g., carboxyls or hydroxyls). Polyamino acid polymers generated synthetically are normally considered to be synthetic if they are not found in nature and are engineered not to be identical to naturally occurring biomolecules.

Some hydrogels are made with a polyethylene glycol-containing precursor. Polyethylene glycol (PEG, also referred to as polyethylene oxide when occurring in a high molecular weight) refers to a polymer with a repeat group (CH2CH2O)n, with n being at least 3. A polymeric precursor having a polyethylene glycol thus has at least three of these repeat groups connected to each other in a linear series. The polyethylene glycol content of a polymer or arm is calculated by adding up all of the polyethylene glycol groups on the polymer or arm, even if they are interrupted by other groups. Thus, an arm having at least 1000 MW polyethylene glycol has enough CH2CH2O groups to total at least 1000 MW. As is customary terminology in these arts, a polyethylene glycol polymer does not necessarily refer to a molecule that terminates in a hydroxyl group. Molecular weights are abbreviated in thousands using the symbol k, e.g., with 15K meaning 15,000 molecular weight, i.e., 15,000 Daltons. SG or SGA refers to succinimidyl glutarate. SS refers to succinate succinimide. SS and SG are succinimidyl esters that have an ester group that degrades by hydrolysis in water. Hydrolytically degradable thus refers to a material that would spontaneously degrade in vitro in an excess of water without any enzymes or cells present to mediate the degradation. A time for degradation refers to effective disappearance of the material as judged by the naked eye. Trilysine (also abbreviated LLL) is a synthetic tripeptide. PEG and/or hydrogels may be provided in a form that is pharmaceutically acceptable, meaning that it is highly purified and free of contaminants, e.g., pyrogens.

### Functional Groups

The precursors have functional groups that react with each other to form the material, either outside a patient, or in situ. The functional groups generally have polymerizable groups for polymerization or react with each other in electrophile-nucleophile reactions or are configured to participate in other polymerization reactions. Various aspects of polymerization reactions are discussed in the precursors section herein.

Thus in some embodiments, precursors have a polymerizable group that is activated by photoinitiation or redox systems as used in the polymerization arts, e.g., or electrophilic functional groups that are carbodiimidazole, sulfonyl chloride, chlorocarbonates, n-hydroxysuccinimidyl ester, succinimidyl ester or sulfasuccinimidyl esters, or as in U.S. Pat. Nos. 5,410,016, or 6,149,931, each of which are hereby incorporated by reference herein in its entirety to the extent they do not contradict what is explicitly disclosed herein. The nucleophilic functional groups may be, for example, amine, hydroxyl, carboxyl, and thiol. Another class of electrophiles are acyls, e.g., as in U.S. Pat. No. 6,958,212, which describes, among other things, Michael addition schemes for reacting polymers.

Certain functional groups, such as alcohols or carboxylic acids, do not normally react with other functional groups, such as amines, under physiological conditions (e.g., pH 7.2-11.0, 37° C.). However, such functional groups can be made more reactive by using an activating group such as N-hydroxysuccinimide. Certain activating groups include carbonyldiimidazole, sulfonyl chloride, aryl halides, sulfosuccinimidyl esters, N-hydroxysuccinimidyl ester, succinimidyl ester, epoxide, aldehyde, maleimides, imidoesters and the like. The N-hydroxysuccinimide esters or N-hydroxysulfosuccinimide (NHS) groups are useful groups for crosslinking of proteins or amine-containing polymers, e.g., amino terminated polyethylene glycol. An advantage of an NETS-amine reaction is that the reaction kinetics are favorable, but the gelation rate may be adjusted through pH or concentration. The NETS-amine crosslinking reaction leads to formation of N-hydroxysuccinimide as a side product. Sulfonated or ethoxylated forms of N-hydroxysuccinimide have a relatively increased solubility in water and hence their rapid clearance from the body. An NETS-amine crosslinking reaction may be carried out in aqueous solutions and in the presence of buffers, e.g., phosphate buffer (pH 5.0-7.5), triethanolamine buffer (pH 7.5-9.0), or borate buffer (pH 9.0-12), or sodium bicarbonate buffer (pH 9.0-10.0). Aqueous solutions of NETS based crosslinkers and functional polymers preferably are made just before the crosslinking reaction due to reaction of NETS groups with water. The reaction rate of these groups may be delayed by keeping these solutions at lower pH (pH 4-7). Buffers may also be included in the hydrogels introduced into a body.

In some embodiments, each precursor comprises only nucleophilic or only electrophilic functional groups, so long as both nucleophilic and electrophilic precursors are used in the crosslinking reaction. Thus, for example, if a crosslinker has nucleophilic functional groups such as amines, the functional polymer may have electrophilic functional groups such as N-hydroxysuccinimides. On the other hand, if a crosslinker has electrophilic functional groups such as sulfosuccinimides, then the functional polymer may have nucleophilic functional groups such as amines or thiols. Thus, functional polymers such as proteins, poly(allyl amine), or amine-terminated di- or multifunctional poly(ethylene glycol) can be used.

One embodiment has reactive precursor species with 3 to 16 nucleophilic functional groups each and reactive precursor species with 2 to 12 electrophilic functional groups each; artisans will immediately appreciate that all the ranges and values within the explicitly stated ranges are contemplated.

The functional groups may be, e.g., electrophiles reactable with nucleophiles, groups reactable with specific nucleophiles, e.g., primary amines, groups that form amide bonds with materials in the biological fluids, groups that form amide bonds with carboxyls, activated-acid functional groups, or a combination of the same. The functional groups may be, e.g., a strong electrophilic functional group, meaning an electrophilic functional group that effectively forms a covalent bond with a primary amine in aqueous solution at pH 9.0 at room temperature and pressure and/or an electrophilic group that reacts by a of Michael-type reaction. The strong electrophile may be of a type that does not participate in a Michaels-type reaction or of a type that participates in a Michaels-type reaction.

A Michael-type reaction refers to the 1, 4 addition reaction of a nucleophile on a conjugate unsaturated system. The addition mechanism could be purely polar, or proceed through a radical-like intermediate state(s); Lewis acids or appropriately designed hydrogen bonding species can act as catalysts. The term conjugation can refer both to alternation of carbon-carbon, carbon-heteroatom or heteroatom-heteroatom multiple bonds with single bonds, or to the linking of a functional group to a macromolecule, such as a synthetic polymer or a protein. Michael-type reactions are discussed in detail in U.S. Pat. No. 6,958,212, which is hereby incorporated by reference herein in its entirety for all purposes to the extent it does not contradict what is explicitly disclosed herein.

Examples of strong electrophiles that do not participate in a Michaels-type reaction are: succinimides, succinimidyl esters, or NETS-esters. Examples of Michael-type electrophiles are acrylates, methacrylates, methylmethacrylates, and other unsaturated polymerizable groups.

### Initiating Systems

Some precursors react using initiators. An initiator group is a chemical group capable of initiating a free radical polymerization reaction. For instance, it may be present as a separate component, or as a pendent group on a precursor. Initiator groups include thermal initiators, photoactivatable initiators, and oxidation-reduction (redox) systems. Long wave UV and visible light photoactivatable initiators include, for example, ethyl eosin groups, 2, 2-dimethoxy-2-phenyl acetophenone groups, other acetophenone derivatives, thioxanthone groups, benzophenone groups, and camphorquinone groups. Examples of thermally reactive initiators include 4, 4′ azobis (4-cyanopentanoic acid) groups, and analogs of benzoyl peroxide groups. Several commercially available low temperature free radical initiators, such as V-044, available from Wako Chemicals USA, Inc., Richmond, Va., may be used to initiate free radical crosslinking reactions at body temperatures to form hydrogel coatings with the aforementioned monomers.

Metal ions may be used either as an oxidizer or a reductant in redox initiating systems. For example, ferrous ions may be used in combination with a peroxide or hydroperoxide to initiate polymerization, or as parts of a polymerization system. In this case, the ferrous ions would serve as a reductant. Alternatively, metal ions may serve as an oxidant. For example, the ceric ion (4+ valence state of cerium) interacts with various organic groups, including carboxylic acids and urethanes, to remove an electron to the metal ion, and leave an initiating radical behind on the organic group. In such a system, the metal ion acts as an oxidizer. Potentially suitable metal ions for either role are any of the transition metal ions, lanthanides and actinides, which have at least two readily accessible oxidation states. Particularly useful metal ions have at least two states separated by only one difference in charge. Of these, the most commonly used are ferric/ferrous; cupric/cuprous; ceric/cerous; cobaltic/cobaltous; vanadate V vs. IV; permanganate; and manganic/manganous. Peroxygen containing compounds, such as peroxides and hydroperoxides, including hydrogen peroxide, t-butyl hydroperoxide, t-butyl peroxide, benzoyl peroxide, cumyl peroxide may be used.

An example of an initiating system is the combination of a peroxygen compound in one solution, and a reactive ion, such as a transition metal, in another. In this case, no external initiators of polymerization are needed and polymerization proceeds spontaneously and without application of external energy or use of an external energy source when two complementary reactive functional groups containing moieties interact at the application site.

### Hydrogels and Hydrogel Formation

In general, the precursors may be combined to make a covalently-cross-linked hydrogel. The hydrogel may comprise a therapeutic agent, or agents, released over a suitable period of time. Hydrogels are made in situ.

When made in situ, the crosslinking reactions generally occur in aqueous solution under physiological conditions. The crosslinking reactions preferably do not release heat of polymerization or require exogenous energy sources for initiation or to trigger polymerization. Formation of hydrogels in situ can result in adherence of the hydrogel to the tissue margins. This polymerization will tend to reduce fluid distribution on injection, thereby reducing undesirable nerve/tissue targeting and providing a matrix for extended therapeutic delivery.

An embodiment is a hydrogel with less swelling. The hydrogel may be generally low-swelling, as measurable by the hydrogel having a weight increasing no more than about 50% upon exposure to a physiological solution in the absence of physical restraints for twenty-four hours relative to a weight of the hydrogel at the time of formation. Swelling may be measured or expressed by weight or volume. Some embodiments swell by weight or by volume no more than about 50%, no more than about 20%, or no more than about 0%; artisans will immediately appreciate that all the ranges and values within the explicitly stated ranges are contemplated, e.g., shrinkage from 10% to 20% (negative swelling), swelling from —10% to no more than 50%. One aspect of swelling is that large changes will increase the pressure on the surrounding tissue and nerves. For instance, filling a interstitial space with a swelling hydrogel will cause the hydrogel to have a height that is not apparent to the user at the time of application and/or gelation. Similarly, swelling (and shrinkage) can create forces on surrounding tissues that promote negative outcomes.

One approach for low-swelling is increase the number of crosslinks or solids content. Increasing in these factors, however, will typically effect the mechanical properties of the gel, with more crosslinks making the gel more brittle but stronger and a higher solids content making the gel stronger. These factors can also increase degradation time and may affect interactions with cells. In some embodiments, to reduce swelling, a precursor is chosen that has a high degree of solvation at the time of crosslinking but subsequently become less solvated and having a radius of solvation that effectively shrinks; in other words, the precursor is spread-out in solution when cross-linked but later contracts. Changes to pH, temperature, solids concentration, and solvent environment can cause such changes; moreover, an increase in the number of branches (with other factors being held effectively constant) will tend to also have this effect. The number of arms are believed to stericly hinder each other so that they spread-out before crosslinking, but these steric effects are offset by other factors after polymerization. In some embodiments, precursors have a plurality of similar charges so as to achieve these effects, e.g., a plurality of functional groups having a negative charge, or a plurality of arms each having a positive charge, or each arm having a functional group of similar charges before crosslinking or other reaction.

Hydrogels described herein can include hydrogels that swell minimally after deposition. Such medical low-swellable hydrogels may have a weight upon polymerization that increases no more than, e.g., about 25%, about 10%, about 5%, about 0% by weight upon exposure to a physiological solution, or that shrink (decrease in weight and volume), e.g., by at least about 5%, at least about 10%, or more. Artisans will immediately appreciate that all ranges and values within or otherwise relating to these explicitly articulated limits are disclosed herein. Unless otherwise indicated, swelling of a hydrogel relates to its change in volume (or weight) between the time of its formation when crosslinking is effectively complete and the time after being placed in in vitro aqueous solution in an unconstrained state for twenty-four hours, at which point it may be reasonably assumed to have achieved its equilibrium swelling state. For most embodiments, crosslinking is effectively complete within no more than about three minutes such that the initial weight can generally be noted at about 15 minutes after formation as Weight at initial formation. Accordingly, this formula is used: % swelling=[(Weight at 24 hours−Weight at initial formation)/Weight at initial formation]*100. The weight of the hydrogel includes the weight of the solution in the hydrogel.

Reaction kinetics are generally controlled in light of the particular functional groups, their concentrations, and the local pH unless an external initiator or chain transfer agent is required, in which case triggering the initiator or manipulating the transfer agent can be a controlling step. In some embodiments, the molecular weights of the precursors are used to affect reaction times. Precursors with lower molecular weights tend to speed the reaction due to their higher concentration of reactive groups, so that some embodiments have at least one precursor with a molecular weight of less than about 1000 or about 2000 Daltons; artisans will immediately appreciate that all the ranges and values within the explicitly stated ranges are contemplated, e.g., from 100 to about 900 Daltons or from 500 to about 1800 Daltons.

The crosslinking density of the resultant biocompatible cross-linked polymer is controlled by the overall molecular weight of the crosslinker and functional polymer and the number of functional groups available per molecule. A lower molecular weight between crosslinks such as 500 will give much higher crosslinking density as compared to a higher molecular weight such as 10,000. The crosslinking density also may be controlled by the overall percent solids of the crosslinker and functional polymer solutions. Increasing the percent solids increases the probability that an electrophilic functional group will combine with a nucleophilic functional group prior to inactivation by hydrolysis. In some methods, to control crosslink density the stoichiometry of nucleophilic functional groups to electrophilic functional groups is adjusted. A one to one ratio leads to the highest crosslink density. Precursors with longer distances between crosslinks are generally softer, more compliant, and more elastic. Thus an increased length of a water-soluble segment, such as a polyethylene glycol, tends to enhance elasticity to produce desirable physical properties. Thus certain embodiments are directed to precursors with water soluble segments having molecular weights in the range of 3,000 to 100,000; artisans will immediately appreciate that all the ranges and values within the explicitly stated ranges are contemplated e.g., 10,000 to 35,000.

The solids content of the hydrogel can affect its mechanical properties and biocompatibility and reflects a balance between competing requirements. A relatively low solids content is useful, e.g., between about 2.5% to about 20%, including all ranges and values there between, e.g., about 2.5% to about 10%, about 5% to about 15%, or less than about 15%.

An embodiment for making a hydrogel in situ in the presence of a therapeutic agent is to combine precursors in an aqueous solution that can be administered with an applicator to the punctum and/or canaliculus and thereafter form the hydrogel. The precursors may be mixed with an activating agent before, during, or after administration. The hydrogel may be placed with a therapeutic agent dispersed therein, e.g., as a solution, suspension, particles, micelles, or encapsulated. Crosslinking, in one embodiment, entraps the agent. In some embodiments, the crosslinking causes the agent to precipitate or move from solution to suspension.

Thus one embodiment relates to combining a first hydrogel precursor with a first type of functional groups with a second hydrogel precursor having a second type of functional groups in an aqueous solvent in the presence of a therapeutic agent in the solvent. In one embodiment, the precursors are dissolved separately and combined in the presence of an activating agent that provides for effective crosslinking. Alternatively, the mere mixing of the precursors triggers crosslinking. Accordingly, one embodiment is providing branched polymer having a plurality of succinimidyl termini dissolved in a low pH (4.0) diluent solution containing a low molecular weight precursor comprising nucleophiles. This solution is activated by combination with a higher pH solution (8.8), initiating the crosslinking mechanism. The agent is pre-loaded as a suspension in the diluent solution. The gel forms in situ.

### Overview of Other Systems

Certain polymerizable hydrogels made using synthetic precursors can, for example, include precursors as used in products such as FOCALSEAL (Genzyme, Inc.), COSEAL (Angiotech Pharmaceuticals), and DURASEAL (Confluent Surgical, Inc.), as in, for example, U.S. Pat. Nos. 6,656,200; 5,874,500; 5,543,441; 5,514,379; 5,410,016; 5,162,430; 5,324,775; 5,752,974; and 5,550,187; each of which are hereby incorporated by reference to the extent they do not contradict what is explicitly disclosed herein. These materials can polymerize too quickly to be injected in a controlled fashion for at least some of the applications described herein. Also, COSEAL and DURASEAL have a high pH, (above pH 9). Another reason is that they apparently swell too much for filling of iatrogenic sites. The swelling of COSEAL and DURASEAL has been measured using an in vitro model in comparison to fibrin sealant (Campbell et al., Evaluation of Absorbable Surgical Sealants: In vitro Testing, 2005). Over a three day test, COSEAL swelled an average of about 558% by weight, DURASEAL increased an average of about 98% by weight, and fibrin sealant swelled about 3%. Assuming uniform expansion along all axes, the percent increase in a single axis was calculated to be 87%, 26%, and 1% for COSEAL, DURASEAL, and fibrin sealant respectively. FOCALSEAL is can swell over 300%. And it also needs an external light to be activated. Fibrin sealant is a proteinaceous glue that has adhesive, sealing, and mechanical properties that are inferior to COSEAL, DURASEAL, and other hydrogels disclosed herein. Further, it is typically derived from biological sources that are potentially contaminated, is cleared from the body by mechanisms distinct from water-degradation, and typically requires refrigeration while stored.

Example Embodiments

An embodiment may include the method of delivering a neuromodulating agent to a nerve, the method introducing a flowable material into a single tissue, or the interstitial space between one or more tissue. The material may be a hydrogel. The material may consist of reactive components for the purpose of forming a hydrogel in situ. The material may comprise a solid suspension of reactive components in an injectable carrier medium, the solid suspension capable of dissolving and reacting in the carrier medium, in the available moisture in the injected tissue, or both. The solid suspension may be a melt, a precipitation, casting or microfluidics. The solid melt particles may be 3D printed or mechanically ground and sieved as such to form monodisperse specific ranges of 20-300 um for the purpose of injection through a 21 g needle.

The particles may contain an agent with neuromodulating properties. The solid reactive precursors may contain an agent in the range of 1 to 75% w/w in proportion, for example between 35 and 50% w/w. The agent has neuromodulating properties. In some instances, the neurolytic properties are for the purpose of ablating a nerve for semi permanent to permanent relief of chronic pain. The agent may be freely incorporated into the solid reactive precursor particles, or incorporated after secondary processing with dissolution enhancers, dissolution inhibitors, or additional agents for extended release such as secondary encapsulation. The material may contain an agent that is highly soluble, have intermediate solubility, or low solubility, wherein the material extends release, decreases burst levels through the delayed onset of hydration through the reactive precursor crosslinking at the solid particle surface. The material may be designed for a bi-modal release through incorporation of freely soluble agent as well as agent for extended release contained within the solid reactive particle dispersion. Multimodal release may also be employed by blending freely available agent in the carrier medium, a more slowly hydrating pre-cross-linked hydrogel particle containing the agent, and the solid reactive melt particles containing the agent. Size ranges of solid reactive melt particles may be blended together to provide various release rates or multimodal release rates, with surface area to volume rations impacting the dissolution rates of the solid reactive precursors and release of the agent contained within. The concentration of pluronic in carrier solutions may also vary to provide various release rates.

The material and application embodiments employ an agent for visualization under medical imaging. The visualization agent is echogenic or radiopaque, or consists of both. The visualization agent in the hydrogel system may be used to confirm sufficient filling of the interstitial space and confirmation of the target tissue. The visualization agent may be used to confirm injection location when the target tissue cannot be visualized and anatomical features used to provide target tissue locations. The material may contain agents for visualization under medical imaging, be suspended in materials containing visualization agents, or contain precursors for the formation of imaging agents on reconstitution. The material may be a suspension that is hyperechoic. The application may uses contrast agent as a carrier medium to provide imaging under fluoroscopy.

The material may comprise a hydrophilic polymer. The material may comprise a polymer comprising the group —(CH2CH2O)—. The material may further comprise a therapeutic agent. The material may be degradable in vivo. The material may be hydrolytically degradable. The material may be degradable in vivo in less than about 3 days to 3 months . The material may contact the nerve for at least one day. The material may be degradable in vivo in more than about one half day and in less than about 90 days. The material, in some instances, may last a minimum of 14 days. The material may be substantially conformable in and around the space of a nerve. The material may partially react outside the tissue and formation of the hydrogel may be completed in the tissue. The material may be formed from at least two chemically distinct precursors that react with each other to form the hydrogel. The at least two precursors may comprise a first precursor having a first functional group and a second precursor having a second functional group, wherein the first functional group reacts with the second functional group to form a covalent bond. The material may be formed from two precursors containing the required functional groups to form covalent bonds but mixed in a single solution, wherein the premixed solution is activated by the introduction of a second solution that accelerates the reaction conditions. The material may be pre-formed by mixing the molten material in an anhydrous environment and may be subsequently suspended in an oil based or aqueous carrier solution at the time of injection thus initiating polymerization. The first functional group may comprise an electrophile and the second functional group may comprise a nucleophile. The electrophile may comprise a succinimide ester. The nucleophile may comprise an amine. In some embodiments, the electrophile is a large molecular weight succinimide ester and the nucleophile is a small molecular weight amine such as trilysine. In some embodiments, both the electrophile and nucleophile are large molecular weight molecules. The A method wherein the first precursor comprises at least three of the first functional group, or at least two, four six, or eight. The second precursor may comprises at least four of the second functional group or at least two, six, or eight. The material and its application may use a large molecular weight first precursor and a low molecular weight second precursor to allow for pre-mixing. The material may be formed from at least one precursor that forms the hydrogel upon exposure to an activation agent, such as an accelerator agent. The at least one precursor may comprise a polymerizable functional group that comprises at least one vinyl moiety prior to exposure to the activation agent. The polymerizable functional group that comprises the at least one vinyl moiety may be, e.g., acrylate, methacrylate, methylmethacrylate. The polymerizable functional group may be polymerizable using free radical polymerization, anionic polymerization, cationic vinyl polymerization, addition polymerization, step growth polymerization, or condensation polymerization. The activation agent may be a polymerization initiator. The material may be formed by at least two polymers with opposite ionic charges that react with each other, a composition of a polymer comprising poly(alkylene) oxide and another polymer that undergoes an association reaction with the polymer comprising poly(alkylene) oxide, a thixotropic polymer that forms the hydrogel after introduction into the tissue, a polymer that from the hydrogel upon cooling, a polymer that forms physical crosslinks in response to a divalent cation, and a thermoreversible polymer. The material may comprise a natural polymer.

An embodiment may include a method of ablating a nerve, the method comprising injecting individual solid melt particles capable of dissolving and reacting in situ for the formation of a larger depot for therapeutic delivery. The solid precursor material may be soluble in carrier medium on start of reconstitution, in tissue post-injection, or both. The material is reactive, with the reactive components at the solid particle to liquid interface reacting first, decreasing dissolution of subsequent solid material. The particles may be visualized under medical imaging via agents incorporated into the particles, the carrier medium, or both. The method involves ablation of a nerve using a neurolytic delivered from the solid particles or neurolytics with secondary encapsulation, with the release kinetics of the neurolytic extended via the reduction of dissolution of further reactive components by those components reacted at the solid particle to liquid interface. The method results in 50%, 75% or >95% ablation of the nerve, with some embodiments of >95%. The method is lasting for a clinically relevant window of 6 months. Some embodiments may include a method for ablating a nerve that lasts >6 months. In some embodiments, the formation of a solid hydrated hydrogel depot from the solid reactrive precursor particle injection forms a solid barrier to nerve regrowth and/or regeneration.

### EXAMPLES

### Example 1

A 10% (w/w) Pluronic solution was formulated by adding x g pluronic to a x-mM borate buffer containing x % trilysine. To this, x mg of amiodarone solid powder was added, obtaining a cloudy suspension (Accelerator Suspension). In a separate solution, 15% PEG-SG was dissolved in monobasic solution (Polymer Solution). The Polymer Solution and the Accelerator Suspension were mixed at a 1:1 ratio and a gel formed in <5 sec. Total curing was allowed to occur x hrs.

### Example 2

Solid powder amiodarone was added to an 85% (v/v) ethanol solution containing PEG-SG. This was mixed 1:1 with a sodium bicarbonate solution to form a gel. The gel was immersed in PBS in which the gel turned cloudy. Release of amiodarone into PBS was measured, and was extended release in nature.

### Example: Non-Reactive Slow Release System

### Carrier Solution “A”:

A 30% (w/w) Pluronic solution in 5×PBS was formulated by adding 3.0 g of F127 polaxomer to 7 ml of 5×PBS and mixing until in solution.

### Particles “A”:

24% (w/w) amitriptyline particles were prepared by combining 1.25 g of amitriptyline powder with 2 g of F127 polaxomer, 1 g of polyvinylpyrrolidone, 0.5 g of 35,000 Da polyethylene glycol (PEG), 0.5 g of 200,000 Da PEG and 0.050 g of sodium tetraborate decahydrate. This powder was melted at 180° C. and mixed until homogenous. The resulting solid was ground in a homogenizer until a powder was obtained. The powder was sieved and particles between 40 μm and 325 μm were collected.

### 200 μl Suspension for Injection “A”:

A 200 μl 35 mg/ml (w/v) amitriptyline slow release injectable suspension was formulated by adding 28 mg of Particles “A” to 172 μl of Carrier Solution “A” and mixing syringe-syringe.

### Preclinical Testing:

Pharmacodynamic testing of Suspension for Injection “A” in a Chronic Constriction Injury (CCI) rat model of neuropathic pain produced a clear trend towards a reduction in allodynic response at 0.5 hrs after dosing and after 4 hr the reduction was significant when compared to blank vehicle tested animals. The reduced allodynic response in treated animals was statistically significant weekly through 5 weeks post dosing.

### Example: In-Situ Polymerizing Slow Release System

### Carrier Solution “B”:

A 15% (w/w) Pluronic solution in phosphate buffer was formulated by adding 1.5 g of F127 polaxomer to 8.5 ml of a 9 mg/ml sodium phosphate monobasic salt solution and mixing until in solution.

### Drug Loaded Particles “B”:

25% (w/w) amitriptyline particles were prepared by combining 2.5 g of amitriptyline powder with 2.5 g 8 arm 20 kDa PEG amine HCl salt, 5.0 g 4 arm 20 kDa PEG succinimidyl glutarate and 0.15 g of dibasic sodium phosphate dihydrate. This powder was melted at 180° C. and mixed until homogenous. The resulting solid was ground in a homogenizer until a powder was obtained. The powder was sieved and particles between 40 μm and 325 μm were collected.

### Bland Particles “C”:

Drug free particles were prepared by combining 2.5 g 8 arm 20 kDa PEG amine HCl salt, 5.0 g 4 arm 20 kDa PEG succinimidyl glutarate and 0.15 g of dibasic sodium phosphate dihydrate. This powder was melted at 180° C. and mixed until homogenous. The resulting solid was ground in a homogenizer until a powder was obtained. The powder was sieved and particles between 40 μm and 325 μm were collected.

### 200 μl Suspension for Injection “B”:

All injectable suspensions were formulated at 17% (w/v) particles in Carrier Solution “B” by adding 34 mg of a blend of particles “B” and “C” to 166 μl of Carrier Solution “B”.

### Doses:

Dose A: 35 mg/ml (w/v) of amitriptyline powder blend contained a blend of 28 mg of Drug Loaded Particles “B” and 6mg of Bland Particles “C” in 166 μl of Carrier Solution “B”.

Dose B: 18 mg/ml (w/v) of amitriptyline powder blend contained a blend of 14 mg of Drug Loaded Particles “B” and 20 mg of Bland Particles “C” in 166 μl of Carrier Solution “B”.

Dose C: 12 mg/ml (w/v) of amitriptyline powder blend contained a blend of 10 mg of Drug Loaded Particles “B” and 24 mg of Bland Particles “C” in 166 μl of Carrier Solution “B”.

Dose D: 7 mg/ml (w/v) of amitriptyline powder blend contained a blend of 6 mg of Drug Loaded Particles “B” and 28 mg of Bland Particles “C” in 166 μl of Carrier Solution “B”.

Dose E: 3.5 mg/ml (w/v) of amitriptyline powder blend contained a blend of 3 mg of Drug Loaded Particles “B” and 31 mg of Bland Particles “C” in 166 μl of Carrier Solution “B”.

### Preclinical Testing:

Suspension for Injection “B” was delivered to a healthy sciatic nerve of rats, and nerves were harvested for histopathologic and pharmacokinetic analysis at days 1, 2 and 7.

Nerve necrosis/degeneration was dose dependent. In the highest dose of the test article (Group A 35 mg/m1) sciatic nerve necrosis/degeneration was at or near 100% at all time points. At 18 mg/m1 the test article induced a mean nerve necrosis of 91.1% at Day 1 (the only time point present for this group). Reducing the test article (TA) dose to 12 mg/ml resulted in a notable decrease in nerve necrosis at Day 1 (45.6%) and Day 2 (38.9%), compared to the two higher doses. By Day 7 nerve necrosis/degeneration increased in this group to a mean of 77.8%. It is possible that analysis of nerve necrosis by H&E staining underestimates the true degree of necrosis, or that nerve necrosis and degeneration continue over time. The 7 mg/ml and 3.5 mg/ml doses of the TA resulted in even less nerve necrosis, in a dose-dependent manner. No obvious difference in nerve necrosis was noted between more heavily and less heavily myelinated axons in any dose group or time point.

The tissue concentrations at 24 hrs were a good fit exponential trendline vs. dose delivered. This line is exponential since the elimination rate of drugs from tissue is an exponential rate (t1/2). The fit of this trendline indicates that there was good dose uniformity across all groups, and that the rate of drug delivery (% of total dose over time) across all groups was very similar.

### Example Embodiments

A pharmaceutically acceptable implant system comprising one or more of the following:
a neuromodulating agent;
solid particulate hydrogel particles;
a carrier medium in a different physical phase; and
in situ formation of a singular depot for sustained release of said agent.

A implant system comprising one or more of the following:
a suspension of particles,
wherein the particles are in a different physical phase than an injectable carrier medium,
wherein the particles contain a neuromodulating agent for sustained delivery to a nerve, and
wherein the system is both echogenic and radiopaque.

An injectable neuromodulating system comprising one or more of the following:
a plurality of solid particles suspended in an injectable carrier medium,
wherein the particles comprise an agent capable of modulating a nerve, and
wherein the particles form a hydrogel depot capable of sustained delivery of the agent.

A process for making an implantable system comprising preparing one or more of the following:
a neuromodulating agent; and
particles in a different physical phase from an injectable carrier medium,
wherein the particles are capable of sustained delivery of the agent, and
wherein the system is both radiopaque and echogenic.

A method for treating chronic pain comprising one or more of the following:
injection particles suspended in a different physical phase from an injectable carrier medium,
wherein the particles are capable of sustained delivery of the agent to a nerve, and
wherein the system can be visualized under ultrasound or fluoroscopy.

A system as described in any embodiment herein, wherein the particles contain reactive precursors.

A system as described in any embodiment herein, wherein reactive precursors covalently bond to form a hydrogel.

A system as described in any embodiment herein, wherein the material includes a hyperechoic agent.

A system as described in any embodiment herein, wherein the hyperechoic agent is entrapped in the particles.

A system as described in any embodiment herein, wherein the agent is a gas.

A system as described in any embodiment herein, wherein the hyperechoic agent are the reactive precursor particles in suspension.

A system as described in any embodiment herein, wherein the hydrogel particles are spheroidal with a maximum diameter of between about 20 to about 300 microns.

A system as described in any embodiment herein, wherein the particles are 50-150 um.

A system as described in any embodiment herein, wherein the particles react to form a large hydrogel depot.

A system as described in any embodiment herein, wherein the hydrogel depot being hydrolytically biodegradable.

A system as described in any embodiment herein, wherein the hydrogel is a product of a covalent crosslinking chemical reaction between at least two precursors, with one of the precursors comprising a branched polyethylene glycol with at least four arms.

A system as described in any embodiment herein, wherein degradation products of the hydrogel particles comprise a polyethylene glycol covalently bound a water labile segment capable of hydrolysis.

A system as described in any embodiment herein, wherein the hydrogel particles, before hydrolysis, have a total swellability in physiological solution of no more than about 10% by volume.

A system as described in any embodiment herein, wherein the implant comprises hydrogel particles and a radiopaque agent.

A system as described in any embodiment herein, wherein the radiopaque agent is contrast medium containing the suspended hydrogel particles.

A system as described in any embodiment herein, wherein the collection having a 18 gauge needle.

A system as described in any embodiment herein, wherein the collection of particles is completely biodegradable at a time between about 30 and about 365 days.

A system as described in any embodiment herein, wherein the collection comprises a plurality of sets of the particles, with the sets having different rates of biodegradation.

A system as described in any embodiment herein, wherein a first set of the particles is biodegradable within about 8 to about 12 days and a second set of the particles is degradable within about 45 to about 55 days.

A system as described in any embodiment herein, further comprising a therapeutic agent.

A system as described in any embodiment herein, further comprising a nerve-active or nerve affecting agent.

A system as described in any embodiment herein, further comprising a neurolytic agent.

A system as described in any embodiment herein, wherein the agent is loaded between 1 and 75% within the particle on a mass basis.

A system as described in any embodiment herein, wherein the agent is released in a sustained manner from the injectable depot.

A system as described in any embodiment herein, wherein the agent is released in a sustained manner due to delayed diffusion of water into a reactive hydrogel particulate system.

A system as described in any embodiment herein, wherein the agent is highly soluble and releases >24 hours.

A system as described in any embodiment herein, wherein the agent is encapsulated in a hydrophobic secondary polymer for suspension within the precursor melt particles.

1.-32. (canceled)

33. A pharmaceutically acceptable implant system comprising:
a neuromodulating agent;
solid particulate hydrogel particles;
a carrier medium in a different physical phase; and
in situ formation of a singular depot for sustained release of said agent.

34. The pharmaceutically acceptable implant system of claims 33, wherein the hydrogel particles contain reactive precursors.

35. The pharmaceutically acceptable implant system of claim 34, wherein reactive precursors covalently bond to form a hydrogel.

36. The pharmaceutically acceptable implant system of claim 33, wherein the hydrogel particles are spheroidal with a maximum diameter of between about 20 to about 300 microns.

37. The pharmaceutically acceptable implant system of claim 36, wherein the hydrogel particles are 50-150 um.

38. The pharmaceutically acceptable implant system of claim 33, wherein the neuromodulating agent may be an anesthetic.

39. The pharmaceutically acceptable implant system of claim 33, further comprising a therapeutic agent.

40. The pharmaceutically acceptable implant system of claim 33, wherein the neuromodulating agent is released in a sustained manner due to delayed diffusion of water into a reactive hydrogel particulate system.

41. A implant system comprising:
a suspension of particles,
wherein the particles are in a different physical phase than an injectable carrier medium,
wherein the particles contain a neuromodulating agent for sustained delivery to a nerve, and
wherein the implant system is both echogenic and radiopaque.

42. The implant system of claim 41, wherein the particles react to form a hydrogel depot.

43. The implant system of claim 42, wherein the hydrogel is a product of a covalent crosslinking chemical reaction between at least two precursors, with one of the precursors comprising a branched polyethylene glycol with at least four arms.

44. The implant system of claim 43, wherein degradation products of the particles comprise a polyethylene glycol covalently bound a water labile segment capable of hydrolysis.

45. The implant system of claim 42, wherein the hydrogel depot being hydrolytically biodegradable.

46. An injectable neuromodulating system comprising:
a plurality of solid particles suspended in an injectable carrier medium,
wherein the particles comprise an agent capable of modulating a nerve, and
wherein the particles form a hydrogel depot capable of sustained delivery of the agent.

47. The injectable neuromodulating system of claim 46, wherein the agent is loaded between 1 and 75% within the particle on a mass basis.

48. The injectable neuromodulating system of claim 46, wherein the agent is released in a sustained manner due to delayed diffusion of water into a reactive hydrogel particulate system.

49. The injectable neuromodulating system of claim 46, wherein the agent is highly soluble and releases >24 hours.

50. The injectable neuromodulating system of claim 46, wherein the agent is encapsulated in a hydrophobic secondary polymer for suspension within precursor melt particles.

51. A method for treating chronic pain comprising:
injection particles suspended in a different physical phase from an injectable carrier medium,
wherein the particles are capable of sustained delivery of an agent to a nerve, and
wherein the particles can be visualized under ultrasound or fluoroscopy.

52. The method of claim 51, wherein the particles comprise a radiopaque agent.
